(19) United States
(12) Patent Application Publication
(10) Pub. No.: US 2022/0009104 A1
(43) Pub. Date: Jan. 13, 2022

(54) ROBOT

(71) Applicant: FRANKA EMIKA GMBH, München (DE)

(72) Inventors: Daniel WAHRMANN LOCKHART, München (DE); Andreas SPENNINGER, Karlsfeld (DE); Mohamadreza SABAGHIAN, München (DE); Christoph JÄHNE, München (DE); Zheng QU, Augsburg (DE); Thore GOLL, München (DE); Ahmed WAFIK, München (DE); Benjamin LOINGER, München (DE); Christoph KUGLER, München (DE); Carles CALAFELL GARCIA, München (DE)

(21) Appl. No.: 17/288,812

(22) PCT Filed: Oct. 28, 2019
(86) PCT No.: PCT/EP2019/079443

§ 371 Date: Apr. 26, 2021

(30) Foreign Application Priority Data
Oct. 26, 2018 (DE) 10 2018 126 873.4

(51) Int. Cl. B25J 9/16; B25J 9/06; B25J 11/00; B25J 13/08; A61B 8/12; A61B 8/00

(52) U.S. Cl. CPC B25J 9/1689; B25J 9/06; B25J 11/008; B25J 13/087; A61B 8/4245; B25J 9/163; B25J 9/1664; A61B 8/12; B25J 9/1633

(57) ABSTRACT

A mobile robot including a mobile base element and at least one multi-jointed manipulator, wherein the robot includes several telemedical devices. The invention also relates to a robot for performing a movement sequence together with a limb of a human with the help of a manipulator.

### FIELD OF THE INVENTION

The present invention relates to a robot configured to interact actively or passively, indirectly or directly, with a human or patient in the course of medical care, therapy, rehabilitation, diagnostics, counseling, etc.

### BACKGROUND OF THE INVENTION

A major focus in the application of robots designed to interact with humans is in the area of care for the elderly or for humans otherwise in need of care. Here, robots, which do not necessarily have to be designed as humanoid robots, are to cooperate with humans, for example in a care center or preferably still at home, by not only helping people with everyday tasks that need to be done in the household, but also supporting people in terms of their ability to move around in order to avoid physically stressful situations. It would make perfect sense for such “care robots” to also take over basic medical work.

Medical robots as such are well known and are mainly used in the field of surgery, whereby these robots always have to be operated by a user, the doctor, for example by means of corresponding input devices.

### SUMMARY OF THE INVENTION

Based on this, it is an object of the present invention to provide a robot that can preferably perform and offer further medical services in addition to implemented assisting activities related to the care and support of humans. Thereby, it is, among other things, an aim of the invention to use robots, which are known in outline from industrial applications in the field of human-robot collaboration (HRC), also in the field of medicine, care, therapy and rehabilitation of humans.

This object is solved with a robot having the features according to claim 1 as well as with a robot having the features according to claim 18.

In a first aspect, the invention proposes a robot comprising a mobile base element and provided with at least one multi-articulated or—joint robot arm or manipulator adapted to interact directly or indirectly with a human, and further comprising at least one telemonitoring device and/or at least one telediagnostic device and/or at least one telemetry device and/or at least one teletherapy device, wherein the robot arm is configured to be compliant-controlled.

This gives the robot arm a fine sensitivity that enables the robot to interact with the human in the intended manner without injuring the human. By interaction in the sense of this invention is meant not only a simple touching of the human being at certain points, as will be explained below in connection with the use of sensors and probes, but also the active guiding of limbs, the supporting guiding together with the movements executed by the human being or even the guiding of the robot arm exclusively by the movements executed by the human being.

Robots with position-controlled axes are fundamentally unsuitable for interaction with a human or patient in the context of the aforementioned touching and common movements, since the forces acting on the robot from outside must be measured for position control, which form the basis for a desired dynamic behavior that is then transferred to the robot via inverse kinematics, also known as admittance control. In the present case, the programming effort would be too high due to the robot's movements at many different positions, which movements are alternating depending on their kind. The required position control would have to be highly accurate, but this is already impossible because the human being himself can move in the course of interaction with the robot and would therefore constantly change his position. Due to the control principle used, such robots are therefore not able to detect such deviations in the course of movement or the movements performed by humans with respect to position and forces in order to react accordingly.

According to the invention, the at least one robot arm, preferably all robot arms of the robot system to be used, shall be equipped with such an integrated compliance control or be equipped with an intrinsic compliance or with a combination of active and passive compliance. In order to be able to perform the operations to be carried out in the course of the desired interaction with a human being, multi-axis HRC robots, preferably of lightweight construction, which can be programmed in such a way with regard to compliance behavior, are to be used for this purpose.

The compliance control is based, for example, on the so-called impedance control, which, in contrast to the already mentioned admittance control, has as its object a real torque control at joint level. Here, depending on a desired dynamic behavior and taking into account the deviations of an actual position from a defined nominal position and/or an actual velocity from a nominal velocity and/or an actual acceleration from a nominal acceleration, forces or torques are determined which are then mapped via the known kinematics of the robot, resulting from the number and arrangement of the joints and axes and thus degrees of freedom, to corresponding joint torques which are set via the torque control. The torque sensor elements integrated in the joints for this purpose detect the one-dimensional torque prevailing in each case at the output of the transmission of the drive unit located in the joint, which can take into account the elasticity of the joint as a measured variable within the scope of the control. In particular, the use of a corresponding torque sensor device, in contrast to the use of only one force torque sensor at the end effector, as in admittance control, also allows the measurement of forces exerted not only on the end effector but on the links of the robot as well as on an object held by or to be manipulated by the robot, such as a probe or the human being or individual limbs themselves, taking into account that human tissue is, moreover, soft and compliant. Torques can also be measured via force sensors in the structure and/or base of the robotic system. In particular, articulation mechanisms between the individual axes of the manipulator can also be used, which allow multi-axis torque detection. Also conceivable are translational joints that are equipped with corresponding force sensors.

The compliance control and sensitivity of the HRC robot realized in this way proves advantageous for the present invention in many respects.

The robot according to the invention, which is preferably designed as a mobile robot so that it can move freely and preferably autonomously in predetermined premises, links in an inventive way areas of telemedicine with its other properties relating to the care and support of humans, which can be set and realized by the implemented compliance behavior or the fine-sensitivity behavior.

Telemedicine is generally understood as diagnostics and therapy bridging a spatial and or temporal distance between a physician (“teledoctor”), therapist, pharmacist, nurse, etc. and a patient. This involves not only remote diagnosis (for example, telecardiology or telediabetology, etc.), but also real-time patient care, for example, teleconsultation, telepsychiatry, teletherapy and telerehabilitation, etc.

In a first embodiment of the invention, the at least one multi-articulated robot arm or manipulator configured to have torque and/or force sensing means in its joints is configured to actively actuate and/or interact with the at least one telemonitoring device and/or the at least one telediagnostic device and/or the at least one telemetry device.

In a preferred embodiment of the robot according to the invention, the telemonitoring device comprises at least one sensor for detecting various vital parameters (such as blood pressure, pulse, ECG, sugar values, etc.), wherein the manipulator is adapted to guide the at least one sensor to a measuring point of the human body corresponding thereto and, in a further step, to place or guide the sensor along there accordingly in order to be able to perform the measurements. This may also include subcutaneous measurements.

In another preferred embodiment of the robot according to the invention, the telediagnostic device comprises at least one ultrasound probe, wherein the robot arm is configured to autonomously guide the probe to a corresponding recording site of a human body and/or along the corresponding recording site while maintaining contact. In addition, the robot arm can be designed to independently change the angle at which the probe is placed on the body, depending on the quality of the recorded images, with a control unit being able, if necessary, to check the information content of the recorded images in real time when the examination is carried out.

In this context, the telemetry device of the robot can be designed to transmit the data (measured values, image data) acquired by means of the sensors and/or the probes to an external receiving point in order to enable, for example, a telemedicine physician to check these data or to allow an implemented monitoring system to initiate appropriate emergency measures in the event of deviations. Thus, the telemetry device can be designed to communicate directly with a WLAN implemented in the premises of the patient to be monitored.

Furthermore, according to the invention, it is provided that the teletherapy device of the robot has an audiovisual device or interface for humans, with the help of which communication between the doctor, nurse, etc. and the patient or person in need of care is possible at any time, in particular also depending on the measurements being performed. This enables not only random communication between the patient or person in need of care and the remotely located doctor or nurse or therapist, but also communication during the performance of measurements or therapeutic steps implemented by means of the robot arm in an appropriate manner. During an ongoing communication, it is even possible according to the invention for the robot arm to make contact with the patient.

Therapies in the sense of the invention may also include the possibility that the robot, by means of its robot arms, may de facto “manually” manipulate the human being or his body parts or his limbs, whether in accordance with a real-time control by the telemedicine physician or therapist or self-controlled, for example in case of an emergency by applying a defibrillator, a syringe or the like, which are directly deposited or arranged on the robot for such emergencies.

The mobile designed robot is designed such that the at least one robot arm has a proximal base (shoulder) and a distal free end (hand), wherein the distal end is configured to automatically grip the sensors and/or probes or to grip a separate end effector that interacts with the sensors and/or probes.

In a preferred embodiment, however, the sensors and/or probes may already be integrally integrated in the distal free end of the robot arm, for example preferably the sensors for measuring blood pressure, pulse or for recording an ECG.

Furthermore, the robot according to the invention can be designed such that a base body or torso is arranged on the mobile base element, at which the proximal base of the manipulator is guided displaceably, in particular linearly. Preferably, a multi-articulated robot arm is guided on each side of the torso via its proximal base in the torso. By means of the mobile base element, the robot itself is freely movable in space and thus relative to a patient. For example, the robot may be configured like a mobile robot as described in German Patent Application No. 10 2016 004 840 A1, the disclosure content of which is expressly referred to herein.

Preferably, a head or head-like device is provided on the torso, which may include the audio-visual device of the teletherapy device, for example a screen or touch screen with a camera and microphone and speakers.

According to the invention, the at least one robot arm is preferably designed as a 7-axis manipulator with realization of corresponding degrees of freedom, which is designed to be correspondingly compliant-controlled and/or force-controlled.

As mentioned above, such a control principle of a robot according to the invention proves to be particularly advantageous with regard to guiding the sensors or probes to a measuring point or recording point on a patient. The compliance control enables a quasi sensitive behavior of the robot arms.

Thus, according to the invention, it is further intended that the guiding of the sensors and/or the probes relative to the measuring/recording point is performed by force-controlled and/or impedance-controlled translational and/or rotational and/or tilting movements of the manipulator. In this way, the robot can virtually “feel” and “sense” the resistances in the point of contact with the human at the recording and measuring points, either through an independent movement or as part of a remote control in which, for example, the telemedicine doctor can check the course and behavior of the robot arm in real time via the camera of the interface. The contact forces that occur can be defined or limited to avoid injury to the patient, for example, by reaching or exceeding at least one predefined threshold condition for a torque acting at the distal end and/or a force acting at the distal end and/or reaching or exceeding an existing or provided force-torque signature and/or a position-velocity signature at the distal end or at the end effector.

Such compliant behavior via different movement patterns, torque patterns and/or force patterns is particularly advantageous with regard to the recording of ultrasound images, since in order to obtain meaningful ultrasound images, the probe must be guided in part with different angular positions relative to the skin at the recording point or also with different force conditions on the skin, which according to the invention the robot can either perform automatically on the basis of its control logic or which can be implemented in real time via a telemedicine doctor.

The robot, which according to the invention is designed to be compliant or sensitive, not only enables interaction with the human being through movements and activities that are performed exclusively on its own, possibly learned through machine learning, but also improves remote control by the telemedicine doctor or therapist.

Due to the fact that the manipulator, via its torque and/or force measurement sensors in its joints, is able to record the resistances, i.e. counter forces and counter torques, that result when a person comes into contact with its effector or with sensors and probes guided by it, these counter forces and counter torques, together with other audiovisually recorded data (such as the patient's answers to questions posed by the telemedicine technician, conveyance of a feeling of pain, recording of facial expressions, etc.) can be transmitted to the telemedicine technician as feedback via a reference manipulator operated by the latter. This reference manipulator is preferably identical in construction to the manipulator on site with the patient and conveys the forces and torques recorded there to the teledoctor through active resistances exerted by the reference manipulator during actuation by the teledoctor, so that the teledoctor can actually feel them. In this way, the doctor himself can “feel” what in turn the manipulator “feels” on site, and act accordingly in real time. The doctor receives tactile feedback, so to speak, on the movements of the robot arm exerted by him.

Whereas in classical remote control systems of a robot arm or manipulator the movements performed by the latter can be controlled visually by a remotely located user via cameras and at most by means of simple haptic feedback signals (vibrations), the design according to the invention enables the forces and torques occurring on site at the patient in the course of the interaction between human and robot arm to be conveyed to the user either directly or, if necessary, by means of a conversion factor (amplification) via the reference, i.e. control, manipulator.

Since the doctor or therapist, by operating the reference manipulator, directly feels what the patient manipulator detects in terms of forces and torques, a doctor can even be enabled to remotely administer injections to the patient, or a therapist can be enabled to manipulate parts of the patient's body via the patient manipulator, e.g. massage, or to guide limbs, as in rehabilitation exercises, as will be explained in connection with a second aspect of the invention.

Since the skin of a human being at the measuring or recording point naturally yields via the soft tissue when the sensors or probes do touch, the use of strictly position-controlled robotic arms, as already mentioned, is fundamentally ruled out for this purpose.

In turn, the compliance control provided in accordance with the invention allows the robot arm to perform controlled movements of its own so that it can, for example, guide the ultrasound probe over and along the intended recording point. In doing so, it is also able to independently sense the different resistances through the soft tissue. Basically, the robot manipulator has to recognize what the actual condition is when the sensor or probe is in contact with the skin, which according to the invention can be realized by appropriate threshold conditions and or individual signatures.

In principle, these signatures are to be understood as concrete characteristic properties of forces and/or torques and/or positions and/or velocities detected at the robot manipulator, which go beyond a simple threshold value. This can include, for example, a specific time behavior of the measured forces, torques, positions and/or velocities, as well as characteristic properties that depend on these parameters.

In another preferred embodiment according to the invention, the robot has at least one control unit that is designed to enable machine learning of the robot as part of the ongoing interaction with the human. By providing appropriate algorithms, the robot is enabled to adapt to the behavior and needs of the human or patient. For example, it can learn over time that the mobility of a limb is increasingly limited, so that when guiding that limb, for example, the robot adjusts its forces and torques according to the resistances generated by the limb.

Furthermore, the robot can also be preset, i.e., programmed, in advance of its use to meet the individual needs and behavior of the human. Thus, the robot according to the invention becomes a personalizable adaptive assistance system for therapy, care, medicine and other support.

In another aspect, the invention relates to a robot having at least one multi-articulated robot arm that is compliant-controlled and configured to be able to perform a predefined sequence of movements intended for the limb when interacting with a limb of a patient.

Such a sequence of movements may, for example, be a rehabilitation exercise. In the context of the present invention, rehabilitation exercise is to be understood as any manipulative measure currently recognized medically, physiotherapeutically, ergo-therapeutically, etc., which can generally be performed and exercised on a patient by a physician, physiotherapist, occupational therapist, etc.

In one embodiment, the robotic arm is configured to perform this sequence of movements or rehabilitative exercise while simultaneously guiding the limb, and preferably with corresponding force-controlled and/or impedance-controlled translational and/or rotational and/or tilting movements.

The limb of the human being can be gripped and held via an e.g. cuff-like holder on the effector, i.e. at the distal end of the multi-axis robot arm, whereby the robot arm, in order to realize the desired sequence of movements, then executes its own controlled movements or reference movements remotely controlled by a therapist via a reference manipulator, and thereby exerts the sequence of movements in terms of force progression and speed on the limb with simultaneous guidance of the latter.

The robot or the at least one robot arm may be arranged on a mobile basis, as in the first-mentioned aspect of the invention, or stationary, for example in the region of a seat or couch. It is also conceivable that the robot arm is arranged on a wheelchair.

In a preferred embodiment of the robot according to the invention, the robot arm is to be designed in such a way that, in the course of the executed sequence of movements, the mobility of the limb can be detected.

In the case of the robot arms used with torque measurement sensors and/or force measurement sensors in the respective joints, any resistances that occur during the performance of a rehabilitation exercise as a result of a lack of mobility of the limb or due to active intervention by the patient can be detected. Due to its compliance control, the robot arm is then able to immediately adjust the further sequence of movements in terms of force, orientation and speed, or to interrupt them or perform them in the reverse order and direction. Such resistances would also be representable in the context of a remote control by a therapist through a reference manipulator.

Preferably, the robot arm shall be configured to determine the degree of mobility of the limb by reaching or exceeding at least one predetermined threshold condition for a torque and/or force acting on the robot arm, and/or reaching or exceeding a predetermined force/torque signature and/or a predetermined position/speed signature on the robot arm.

The sequences of movements in the context of, for example, rehabilitation exercises vary depending on the training condition or daily form of the patient, so that, as a rule, no predetermined strict movements can be performed by the robot arm. Accordingly, in a further embodiment according to the invention, the robot can have at least one control unit that is designed to enable machine learning of the robot, for example with respect to possible sequences of movements to be performed, as part of the interaction with the human.

In further embodiments, the robot arm or manipulator according to the second aspect of the invention may also comprise at least one telemonitoring device and/or at least one telediagnostic device and/or at least one telemetry device and/or at least one teletherapy device.

According to the invention, the compliance behavior that can be realized as a result of impedance control allows a robot or robot arm or manipulator in the above-mentioned embodiments, whether it is remotely controlled by a doctor or therapist or whether it interacts with a human being or patient by means of programmable and learnable movements of its own, to interact with a human or patient always in such a way that the forces (and torques) exerted on the human being by the robot arm in soft areas or limbs can never lead to injury. Due to its sensor technology and due to its impedance control, the robot arm is always able to detect and recognize the compliance of soft tissue (e.g.

examinations of organs via the abdominal wall) or muscle limits and joint limits defining the mobility of a limb when guiding and moving this limb (e.g. during rehabilitation exercises).

### BRIEF DESCRIPTION OF THE DRAWINGS

Further advantages and features of the invention will be apparent from the description of the embodiments illustrated with reference to the accompanying drawings, in which:

FIG. 1 is a schematic view of a robot according to a first aspect of the invention;

FIG. 2 is a perspective view of a mobile robot according to the invention;

FIG. 3 is a schematic view of a mobile robot interacting with a human;

FIG. 4 is a schematic view of an arrangement for remote control between a patient-side robot arm and a reference robot arm;

FIG. 5 is a schematic view of a robot according to a second aspect of the invention; and

FIG. 6 shows a robot arm attached to a wheelchair.

### DETAILED DESCRIPTION

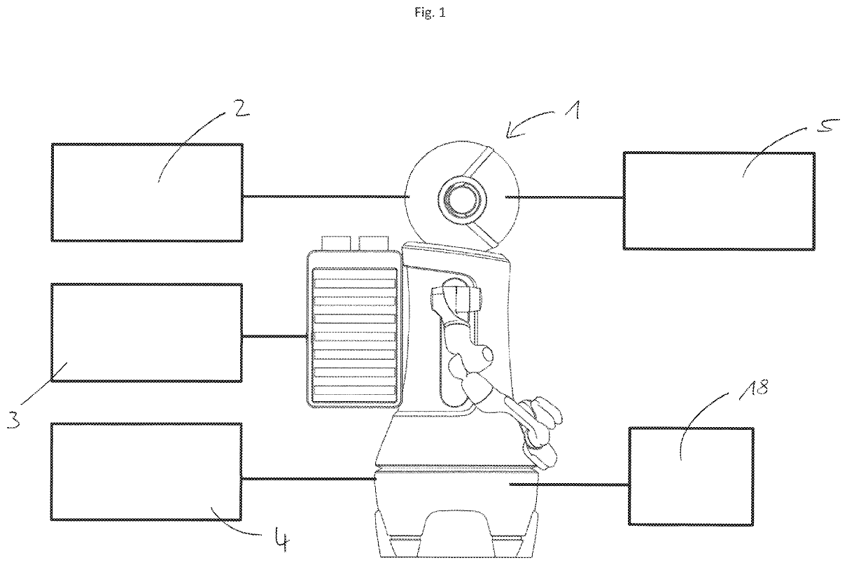

FIG. 1 shows the principle of a mobile robot 1 according to the invention, which can be used, for example, as a care and/or service robot in a patient's household.

The mobile robot 1 is configured to have at least one telemonitoring device 2 and/or at least one telediagnostic device 3 and/or at least one telemetry device 4 and/or at least one teletherapy device 5, each depending on the need having different embodiments. Furthermore, the robot 1 may comprise at least one control unit 18 configured to enable machine learning of the robot 1.

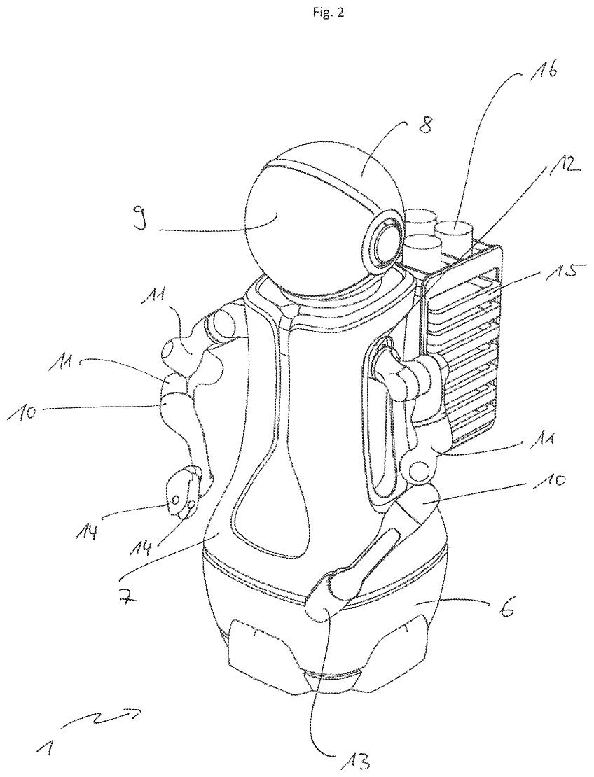

As shown in FIG. 2, the mobile robot 1 comprises a mobile base element 6, which serves as a mobile platform by means of which the robot 1 moves on a plane. For this purpose, motor-driven wheels (not shown) can be arranged within the base element 6.

A torso 7 is located on the mobile base element 6, which can be arranged to rotate about its longitudinal axis relative to the base element 6. On the torso 7 there is also a head 8 which can be arranged rotatably relative to the torso 7.

The head 8 has an interface 9 in the form of a screen with integrated camera and speakers. Via this interface 9, any communication with the outside world is possible, e.g. in the context of video telephony.

On both sides of the torso 7, robot arms or manipulators 10 are provided, which consist of several axis members 11 connected to each other in an articulated manner. The number of axis members 11 or joints defines the total number of degrees of freedom provided by such a manipulator 10.

In accordance with the invention, these robot arms 10 are controlled such that they are compliant and sensitive.

Each manipulator 10 has a proximal base 12 disposed on the torso 7 and a free distal end 13, for example a hand-like gripping mechanism.

The proximal base 12 is linearly slidably movable relative to the torso 7 in the longitudinal direction thereof, namely the proximal base 12 of each manipulator 10 separately.

According to the invention, at least one sensor 14 is integrated in the hand 13, which is designed to detect various vital parameters at a corresponding measuring point of the body or skin upon contact with a human being.

A tray 15 is provided on the rear side of the torso 7, in which, for example, further sensors, in particular an ultrasound probe 16, or emergency devices, such as defibrillators, are located.

The compliant-controlled design of the robot arms 10 according to the invention allows the sensors 16 to be gripped directly by the free distal grippers 13 and guided to the patient. That is, the axis members 11 are configured, dimensioned, and articulated relative to each other and actuatable to permit the manipulators 10 to move such that their distal free end 13 can be moved directly toward the lateral, ventral, and/or dorsal regions or surfaces of the torso 7. The mobility of the manipulators also allows objects to be picked up directly from the floor in front of or behind the robot and laterally thereto, as well as to reach almost any part of a patient who is lying or sitting, for example, to perform the measurement procedures.

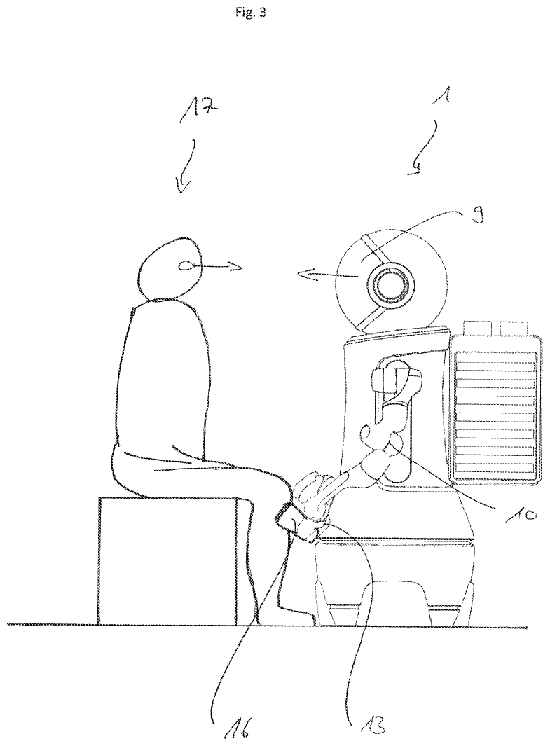

FIG. 3 schematically shows an example of an interaction of the robot 1 according to the invention with a human 17.

The robot 1 uses its robot arm 10 to guide an ultrasound probe 16 to the knee of the seated human 17 by means of its distal hand 13 in order to perform corresponding recordings there, whereby during the ultrasound examination the human 17 can be in direct video and voice contact with a physician via the interface 9.

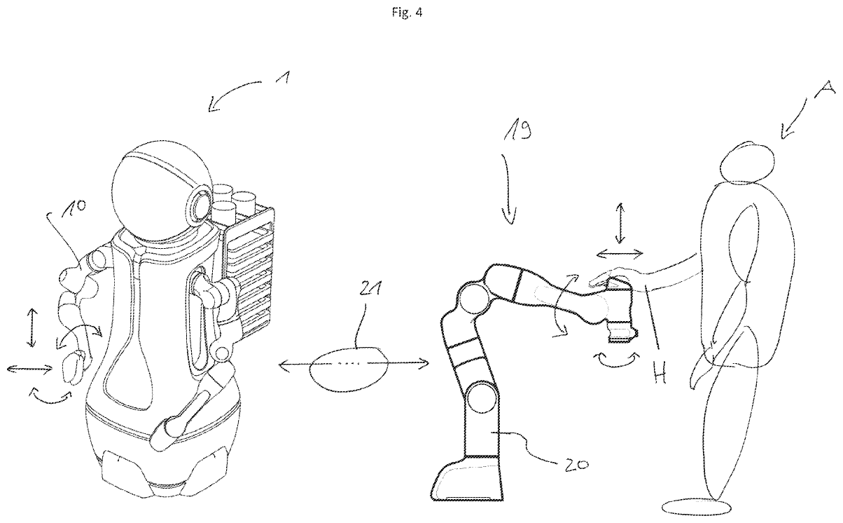

However, this movement can also be remotely controlled by a physician by operating a reference manipulator or robot arm, as illustrated in FIG. 4.

FIG. 4 schematically illustrates the possibility according to the invention to remotely control the robot 1 or at least one robot arm 10 for the purpose of a medical or other therapeutic application.

While the robot 1 is on site at the patient, a reference or control robot 19 is located at the site of the doctor A. The control robot 19 has a reference manipulator 20 that is identical in construction to the robot arm 10 of the patient-side robot 1. In other words, the doctor-side reference manipulator 20 has the same number of degrees of freedom, as well as identical drive units in the joints including torque and force sensors.

The doctor A operates the reference manipulator 20 by guiding it accordingly with his hand H, whereby the movements introduced or applied by the doctor A to the reference manipulator 20 are translated in terms of their quality and quantity into corresponding movements of the robot arm 10, which is symbolized by the arrows in FIG. 4.

In other words, the doctor A performs a movement, for example to guide a probe to a part of the patient's body to be examined, and the forces and torques occurring in the reference manipulator 20 in the process, which in their entirety ultimately define a defined sequence of movements, are transmitted identically to the robot arm 10, whereby the doctor A can monitor and control the course of the examination in real time via the audiovisual means provided in the robot 1 (camera, microphone).

According to the invention, however, a substantial feedback is provided to the doctor A in that the resistances occurring in the course of the movement of the robotic arm 1 during an interaction with the human being, in this case, for example, during the placement of the probe on the soft body part, are detected by the corresponding sensors in the joints of the robot arm 10 as a result of counter forces and counter torques and are transmitted in an identical manner in real time to the reference manipulator 20, which conveys them to the doctor A by corresponding activation of its own drive units in the joints, while the doctor A moves or guides the reference manipulator 20, which is also to be indicated by the arrows. Consequently, the doctor A can feel these resistances himself, and thereby align his further behavior with them and adapt the further sequences of movements.

During the transmission of forces and torques between the reference manipulator 20 on the doctor's side and the robot arm 10 on the patient's side, which takes place via corresponding local or global networks 21 (WLAN, 5G, etc.), predetermined conversion factors (amplification or reduction of the forces) can be used in both directions under certain circumstances.

It can also be provided that the robot 1 is individually adapted to the patient, which has been machine-learned in advance by a patient-specific programming or in the course of a longer interaction by the robot 1, whereby defined threshold conditions are created, which are formed, for example, in the form of force, torque, position and/or speed signatures and/or parameters. In this way, it can be prevented that an incorrect operation on the reference manipulator 20 by the doctor A, which would for example lead to an excessive application of the probe, can cause pain or injury to the patient during the operation and exercise of the telemedical application. Also, such threshold conditions can be used that the robot arm 10 applies the correct, appropriate forces and torques when interacting with the human being, which it knows via experience values as a result of machine learning, adapting, so to speak, the movements pre-set by the doctor A in their quantity, although the latter erroneously operates the reference manipulator 20 in this respect from the beginning. Consequently, according to the invention, the robot 1, as an adaptive assistance system, is capable of correcting the commands of the doctor A when necessary.

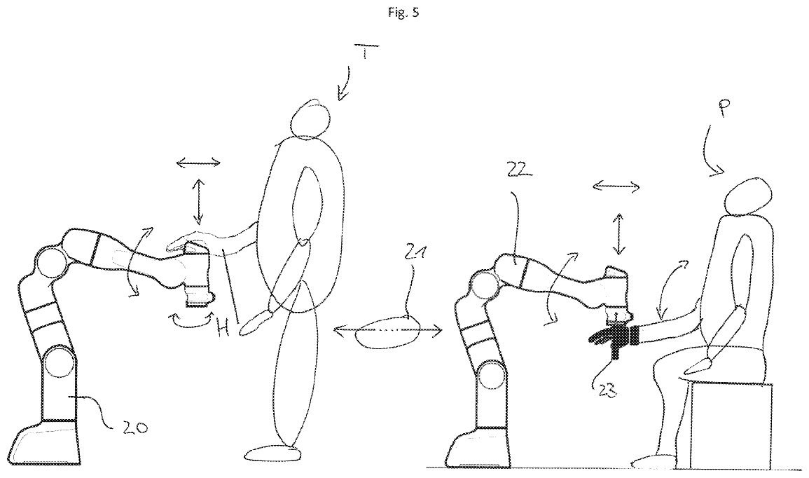

According to the invention, the aforementioned properties and conditions can also be applied to actions in the context of telerehabilitation, in which a defined sequence of movements, reflecting for example a known rehabilitation exercise, is controlled remotely, as illustrated in FIG. 5.

A therapist T operates a reference manipulator 20 remotely (via a network 21). A manipulator 22 of identical configuration is provided at the patient P, which can hold and thus guide an arm of the patient P via its end effector and corresponding ergonomic means, e.g. a cuff 23.

The sequence of movements exerted by the therapist T on the reference manipulator 20 by means of hand H is transferred to the patient-side manipulator 22 identically or taking into account predetermined conversion factors by detecting the forces and torques occurring in the process, i.e. its drive units in the joints are controlled in such a way that this sequence of movements is implemented in a corresponding manner when guiding the arm of the patient P, as indicated schematically by the arrows.

As mentioned above, the therapist T can receive feedback on the resistances occurring in the patient P, and threshold conditions can also be taken into account in order to prevent injuries.

In a preferred embodiment of the invention, however, there is no need for remote control, i.e. specification by a therapist T, but the robot arm 22 is designed in such a way and is already capable itself of being able to perform predetermined sequences of movements within the scope of guiding a limb of a patient P.

Such movement sequences can be stored in a memory of a corresponding control unit, be individually adapted to the patient P or his clinical picture by corresponding pre-programming and/or be further modified and individualized by machine learning.

By guiding the arm of patient P, for example, in the course of a rehabilitation exercise, the robot arm 22 can meanwhile immediately detect the resistances arising in the course of guiding the arm, which may be of a muscular nature and/or specific to the joint, for example, or generated directly by patient P due to pain, via the force measurement and torque measurement sensors in its drive units in the individual joints, and stop, adjust or reverse the further movement. In other words, the detection of resistances that can be mapped as counter forces and/or counter torques can be used as a measure for assessing the mobility.

This is also possible if the patient P moves the robot arm 22 automatically as part of a rehabilitation movement or training sequence performed by the patient P, wherein the robot arm 22 is in a gravity-compensated state, can therefore be guided without resistance, and senses the forces and torques generated by the patient P's movement.

In this regard, the robot arm 22 can also serve as a type of training device for muscle development and mobility. The robot arm 22 is programmed so that when the patient P performs defined sequences of movements, the robot arm 22 opposes the patient P with a defined resistance, which can also change during the exercise. Such resistances or resistance curves can be pre-programmed individualized to the patient P and/or determined over several exercise units by machine learning by the robot itself, since the robot according to the invention is able to detect the respective forces and torques at any time.

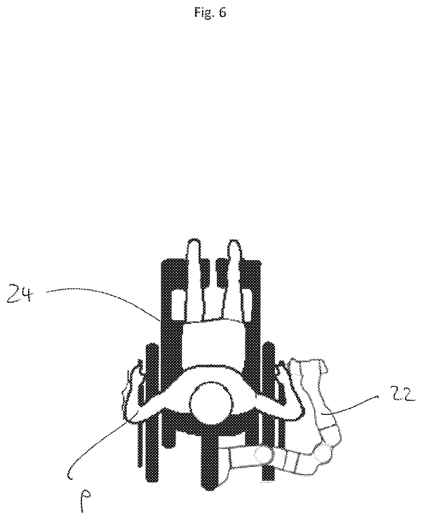

Such a robot arm 22 can be arranged on a mobile platform, stationary or, for example, also on a wheelchair 24, as shown in FIG. 6.

According to the invention, all of the aforementioned embodiments and application examples have in common that both the patient-side manipulator 10,22 and a doctor- or therapist-side manipulator 20 are designed, configured and programmed as a compliant-controlled and thus sensitive robot arm. The robots, as well as the systems in which they are embedded, are preferably designed as machine-learning systems.

1. A robot with a mobile base element and with at least one multi-articulated robot arm, which is designed to interact directly or indirectly with a human being, as well as with at least one telemonitoring device and/or at least one telediagnostic device and/or at least one telemetry device and/or at least one teletherapy device, wherein the robot arm is designed to be compliant-controlled.

2. The robot according to claim 1, wherein said at least one multi-articulated robot arm is adapted to actuate and/or to cooperate with said at least one telemonitoring device and/or at least one telediagnostic device and/or at least one telemetry device and/or at least one teletherapy device.

3. The robot according to claim 1, wherein the telemonitoring device comprises at least one sensor for detecting vital parameters and wherein the robot arm is adapted to guide the sensor to a corresponding measuring point of a body.

4. The robot according to claim 1, wherein the telediagnostic device comprises at least one ultrasound probe and wherein the robot arm is adapted to guide the probe to a corresponding recording site of a body and/or along the corresponding recording site.

5. The robot according to claim 3, wherein the telemetry device is adapted to transmit the data detected by means of the sensors to an external receiving point.

6. The robot according to claim 1, wherein the teletherapy device comprises an audio-visual device.

7. The robot according to claim 1, wherein the at least one robot arm has a proximal base and a distal free end.

8. The robot according to claim 7, wherein the telemonitoring device comprises at least one sensor for detecting vital parameters, wherein the robot arm is adapted to guide the sensor to a corresponding measuring point of a body, and wherein the distal end is adapted to grip the sensors.

9. The robot according to claim 7, wherein the telemonitoring device comprises at least one sensor for detecting vital parameters, wherein the robot arm is adapted to guide the sensor to a corresponding measuring point of a body, and wherein the distal end integrally comprises the sensors.

10. The robot according to claim 7, wherein a torso is provided which is arranged on the mobile base element, at which the proximal base of the robot arm is guided displaceably.

11. The robot according to claim 10, wherein a head is provided on the torso.

12. The robot according to claim 11, wherein the teletherapy device is provided in the torso and/or in the head.

13. The robot according to claim 3, wherein the robot arm is configured such that the guiding of the sensors relative to the measuring/recording point is performed by force-controlled and/or impedance-controlled translational and/or rotational and/or tilting movements.

14. The robot according to claim 13, wherein the robot arm is configured to define a contact force in the region of the measuring/recording point by achieving or exceeding at least one of a predetermined threshold condition for a torque acting at the distal end and/or a force acting at the distal end, and/or achieving or exceeding a provided force/torque signature and/or a position/velocity signature at the distal end.

15. The robot according to claim 13, or wherein the robot arm is remotely controllable.

16. The robot according to claim 10, wherein the telemonitoring device comprises at least one sensor for detecting vital parameters, wherein the robot arm is adapted to guide the sensor to a corresponding measuring point of a body, and wherein the sensors can be deposited on or in the torso.

17. The robot according to claim 1, wherein the robot comprises at least one control unit which is designed to enable machine learning of the robot in the context of interaction with humans.

18. A robot with at least one multi-articulated robot arm, which is controlled for compliance and designed to perform a predefined sequence of movements intended for a limb when interacting with the limb of a human being.

19. The robot of claim 18, wherein the robot arm is configured to perform said sequence of movements while simultaneously guiding the limb.

20. The robot according to claim 19, wherein said robot arm is configured to perform said sequence of movements by force-controlled and/or impedance-controlled translational and/or rotational and/or tilting movements.

21. The robot according to claim 18, wherein the robot arm is configured to detect the mobility of the limb while performing the sequence of movements.

22. The robot according to claim 21, wherein the robot arm is configured to determine the degree of mobility of the limb by at least one of reaching or exceeding a predetermined threshold condition for a torque and/or force acting on the robot arm, and/or reaching or exceeding a predetermined force/torque signature and/or a predetermined position/speed signature on the robot arm.

23. The robot according to claim 18, wherein the robot arm is remotely controllable.

24. The robot according to claim 18, wherein the robot comprises at least one control unit configured to enable machine learning of the robot in the context of interaction with humans.

25. The robot according to claim 18, wherein the robot comprises at least one telemonitoring device and/or at least one telediagnostic device and/or at least one telemetry device and/or at least one teletherapy device.

26. The robot according to claim 4, wherein the telemetry device is adapted to transmit the data detected by means of the probes to an external receiving point.

27. The robot according to claim 7, wherein the telediagnostic device comprises at least one ultrasound probe, wherein the robot arm is adapted to guide the probe to a corresponding recording site of a body and/or along the corresponding recording site, and wherein the distal end is adapted to grip the probes.

28. The robot according to claim 7, wherein the telediagnostic device comprises at least one ultrasound probe, wherein the robot arm is adapted to guide the probe to a corresponding recording site of a body and/or along the corresponding recording site, wherein the distal end integrally comprises the probes.

29. The robot according to claim 4, wherein the robot arm is configured such that the guiding of the probes relative to the measuring/recording point is performed by force-controlled and/or impedance-controlled translational and/or rotational and/or tilting movements.

30. The robot according to claim 10, wherein the telediagnostic device comprises at least one ultrasound probe, wherein the robot arm is adapted to guide the probe to a corresponding recording site of a body and/or along the corresponding recording site, and wherein the probes can be deposited on or in the torso.
